(19) United States
(12) Patent Application Publication
(10) Pub. No.: US 2021/0077585 A1
(43) Pub. Date: Mar. 18, 2021

(54) METHOD OF TREATMENT OF IMMUNE CHECKPOINT INHIBITOR-RELATED IMMUNE ADVERSE EFFECTS

(71) Applicants: Enrico GARACI, (US); Luigina ROMANI, (US)

(72) Inventors: Giorgia RENGA, Perugia (IT); Marina Maria BELLET, Perugia (IT); Marilena PARIANO, Perugia (IT); Claudio COSTANTINI, Perugia (IT); Luigina ROMANI, Perugia PG (IT); Enrico GARACI, Rome (IT)

(21) Appl. No.: 17/013,121

(22) Filed: Sep. 4, 2020

(30) Foreign Application Priority Data
Sep. 13, 2019 (IT) 102019000016310

(51) Int. Cl. A61K 38/22; A61K 39/395; A61P 1/00; A61P 37/02

(52) U.S. Cl. CPC A61K 38/2292; A61K 39/3955; A61K 2039/505; A61P 37/02; A61P 1/00

(57) ABSTRACT

A method for treatment and/or reduction of occurrence of immune checkpoint inhibitor related immune adverse effects in a subject in need thereof, includes administering thymosin alpha 1 to the subject. The immune checkpoint inhibitor related immune adverse effects can include colitis, diarrhea, rash, elevated alanine amino transferase (ALT), hypothyroidism, or hypophysitis.

### FIELD

The present disclosure concerns Thymosin alpha 1 (Tα1) for use in the reduction of occurrence or likelihood of and treatment of immune checkpoint inhibitor related immune adverse effects. In particular, the present disclosure concerns Thymosin alpha 1 for use in the reduction of occurrence or likelihood of and treatment of immune checkpoint inhibitor related immune adverse effects such as checkpoint inhibitor colitis.

### BACKGROUND

Immune checkpoint inhibition is a recently introduced, innovative form of cancer immunotherapy, which aims at removing inhibitory co-stimulatory signals from T cells, mainly tumor-specific cytotoxic CD8+cells, via blockade of cytotoxic T-lymphocyte associated protein-4 (CTLA-4), and/or programmed death protein-1 (PD-1)/PD-ligand 1 (PD-L1) (Pardoll, 2012). The physiological role of such proteins is to restrain the immune system from mounting inappropriate T cell responses; nevertheless, this homeostatic mechanism may be exploited by malignant cells to escape immunological surveillance (Fritz and Lenardo, 2019). It follows that administration of monoclonal antibodies that target CTLA-4, PD-1 and PD-L1 restores the cytotoxic function of lymphocytes and induces effective antineoplastic responses (Wilky, 2019).

To date there are 7 approved checkpoint inhibitors that target 3 main checkpoints, including cytotoxic T-lymphocyte associated protein 4 (CTLA-4; ipilimumab and tremelimumab), programmed cell death receptor 1 (PD-1; pembrolizumab and nivolumab), and programmed death ligand 1 (PD-L1; atezolizumab, avelumab, and durvalumab) (Darvin et al., 2018). However, elimination of immunoregulatory control by those inhibitory pathways may lead to unrestrained activation of effector immune responses resulting in the so-called immune-related adverse effects (irAEs) (Haanen et al., 2018; Ladak and Bass, 2018; Marin-Acevedo et al., 2019). Thus, while representing a remarkable breakthrough in the treatment of several advanced malignancies, several ICI-related adverse events that affect multiple body systems (Table 1) (Samaan et al., 2018) have been recognized, including checkpoint inhibitor-mediated colitis (CIC) and enteritis (Karamchandani and Chetty, 2018; Marin-Acevedo et al., 2018). The incidence of CIC ranges from 1% to 20% depending on the type of checkpoint inhibitors and may be associated with other irAEs. Table 1 shows the percentage ranges of all grade immune-related common adverse events by checkpoint inhibitor class.

TABLE 1

| Class of immune | | | | | | | |
|---|---|---|---|---|---|---|---|
| checkpoint | | | | | | | |
| inhibitors | Approved agents | Rash | Diarrhea | Colitis | Elevated ALT | Hypothyroidism | Hypophysitis |
| Anti | Ipilimumab, | 12%-68% | 31%-49% | 7%-11.6% | 3%-9% | 4%-4.2% | 4%-6% |
| CTLA-4 | Tremelimumab | | | | | | |
| Anti | Nivolumab, | 11.7%-24% | 2.9%-11.5% | 1.3%-2.9% | 1.8%-7.1% | 3.4%-8.5% | 0.25% |
| PD-1 | Pembrolizumab | | | | | | |
| Anti | Atezolizumab, | 7.4% | 11.6%-23% | 0.7%-19.7% | 0.9%-4.0% | 5.0%-9.6% | 0.2% |
| PD-L1 | Durvalumab, | | | | | | |
| | Avelumab | | | | | | |

Notes to Table 1:
CTLA-4: Cytotoxic T-lymphocyte-associated antigen 4; PD-1: Programmed cell death protein 1; PD-L1: Programmed death-ligand 1; ALT: Alanine aminotransferase.

CIC typically occurs 5 weeks-10 weeks after the 2nd or 3rd dose of treatment. Optimal management of CIC requires early recognition and timely use of corticosteroids. About one third to two thirds of patients are steroid refractory. Infliximab is the second line therapy in these patients. Recent reports have shown that Vedolizumab is more gut specific and efficacious in steroid and infliximab refractory cases. Fecal microbiota transplant has recently been reported to be successful in steroid refractory cases. Thus, addressing CIC irAEs has become a major clinical issue for physicians and patients alike (Rocha et al., 2019; Samaan et al., 2018).

In the light of the above, it is therefore apparent the need to provide new therapies to prevent or treat irAEs by checkpoint inhibitor class, in particular checkpoint inhibitor-mediated colitis (CIC) and enteritis.

It is known that Thymosin alpha 1 (Tα1) is a naturally-occurring polypeptide of 28 amino acid first described and characterized by Goldstein et al. in 1972 (Goldstein et al., 1972). Tα1 is well known in the medical field for its immunoregulatory properties in several in vitro and in vivo assay. Previous use of Tal is already known. The peptide has been used worldwide as an adjuvant or immunotherapeutic agent to treat disparate human diseases, including viral infections, immunodeficiencies, and malignancies (Camerini and Garaci, 2015; Garaci et al., 2015; Goldstein and Goldstein, 2009; Li et al., 2015). The peptide can enhance T-cell, dendritic cell and antibody responses, modulates cytokine and chemokine production and blocks steroid-induced apoptosis of thymocytes. Its central role in modulating dendritic cell function and activating multiple signaling pathways differentially contributing to different functions may offer a plausible explanation for its pleiotropic action. Additionally, the ability to activate the indoleamine 2,3-dioxygenase (IDO) 1 enzyme conferring immune tolerance and restraining the vicious circle that perpetuates chronic inflammation has been a turning point, suggesting a potential, specific function in immune-mediated diseases (Romani et al., 2006).

### SUMMARY

According to the present disclosure, it has now been found that Tα1 has the ability to counteract immune-related common adverse events by checkpoint inhibitor class.

In particular, according to the present disclosure, it has been found that Tα1 is able to reduce the likelihood or occurrence of, and even prevent, CIC irAEs in murine models of IDB. Furthermore, according to the present disclosure it has been shown in murine models of CIC that the treatment with Tα1 significantly increased the survival of mice, being the majority of mice surviving at 16 days post initiation of colitis, at the time at which untreated mice have all died. In addition, according to the present disclosure, it has been shown that the treatment with Tα1 does not affect the antitumoral efficacy afforded by treatment with anti-CTLA-4. These results indicate that Tα1 can be advantageously used to ameliorate the immunopathology associated with checkpoint inhibitor blockade.

It is therefore a specific embodiment of the present disclosure to administer thymosin alpha 1 for use in the treatment and/or prevention of immune checkpoint inhibitor related immune adverse effects.

In some embodiments according to the present disclosure, the immune checkpoint inhibitor related immune adverse effects can be selected from the group consisting of checkpoint inhibitor colitis, and other immune adverse effects which are based on the same immune-toxicity mechanism such as diarrhea, rash, elevated alanine amino transferase (ALT), hypothyroidism, and hypophysitis.

Some embodiments of the present disclosure also concern a pharmaceutical composition comprising or consisting of thymosin alpha 1, as active principle, together with one or more excipients and/or adjuvants, for use in the treatment and/or prevention of immune checkpoint inhibitor related immune adverse effects.

As mentioned above, the immune checkpoint inhibitor related immune adverse effects can be selected from the group consisting of checkpoint inhibitor colitis, diarrhea, rash, elevated alanine amino transferase (ALT), hypothyroidism, and hypophysitis.

A further embodiment of the present disclosure is a combination comprising or consisting of Thymosin alpha 1 with one or more immune checkpoint inhibitors for simultaneous, separate or sequential use in the treatment and/or prevention of immune checkpoint inhibitor related immune adverse effects.

As mentioned above, the immune checkpoint inhibitor related immune adverse effects can be selected from the group consisting of checkpoint inhibitor colitis, diarrhea, rash, elevated alanine amino transferase (ALT), hypothyroidism, and hypophysitis.

Said one or more immune checkpoint inhibitors can be selected from the group consisting of anti CTLA-4, anti PD-1 and/or anti PD-L1.

According to the present disclosure, “simultaneous use” is understood as meaning the administration of the two compounds of the combination according to the disclosure in a single and identical pharmaceutical form.

“Separate use” is understood as meaning the administration, at the same time, of the two compounds of the combination according to the disclosure in distinct pharmaceutical forms.

“Sequential use” is understood as meaning the successive administration of the two compounds of the combination according to the disclosure, each in a distinct pharmaceutical form.

The present disclosure now will be further elaborated by an illustrative, but not limitative way, according to certain embodiments thereof, with particular reference to the enclosed drawings.

### BRIEF DESCRIPTION OF FIGURES

FIG. 1 shows the protective effects of Tα1 in murine dextran sodium sulfate (DSS)-induced colitis. A) The panel shows the experimental protocol. Effect of Tal on A) the weight, daily recorded; B) survival; C) colon inflammatory pathology; E) gene expression of indoleamine-3-deoxygenase (IDO)1 and production of IL-10 and F) production of inflammatory IL-17A and IL-1b of mice with DSS. Results in panels D, E and F were obtained at time of sacrifice. None, untreated, naïve mice. **P<0.01 and ***P<0.001 Tal-treated vs untreated mice.

FIG. 2 shows the protective effects of Tα1 in murine model of checkpoint inhibitor-mediated colitis (CIC). A) The panel shows the experimental protocol. Effect of Tα1 on A) the weight, daily recorded; B) survival and C) colon inflammatory pathology and E) tumor growth in mice with CIC (DSS+anti-CTLA treatment).

### DETAILED DESCRIPTION

### Example 1: Treatment of DSS Colitis and Checkpoint Inhibitor-Mediated Colitis (CIC) Murine Models with Thymosin Alpha1 According to an Embodiment of the Present Disclosure

Materials and Methods

Mice.

Inbred C57BL6 mice, 8 to 12 weeks old, were purchased from Charles River Breeding Laboratories (Calco, Italy). Experiments were performed following protocols approved by the institutional animal committee and in accordance with European Economic Community Council Directive as well as institutional animal care and use guidelines.

Thymosin Alpha1.

Tα1 was from CRIBI Biotechnology, Padova Italy. Tα1 and the scrambled polypeptide were supplied as purified (the endotoxin levels were <0.03 pg/ml, by a standard limulus lysate assay) sterile, lyophilized, acetylated polypeptide. The sequences were as described (Romani et al., 2017).

Dss Colitis.

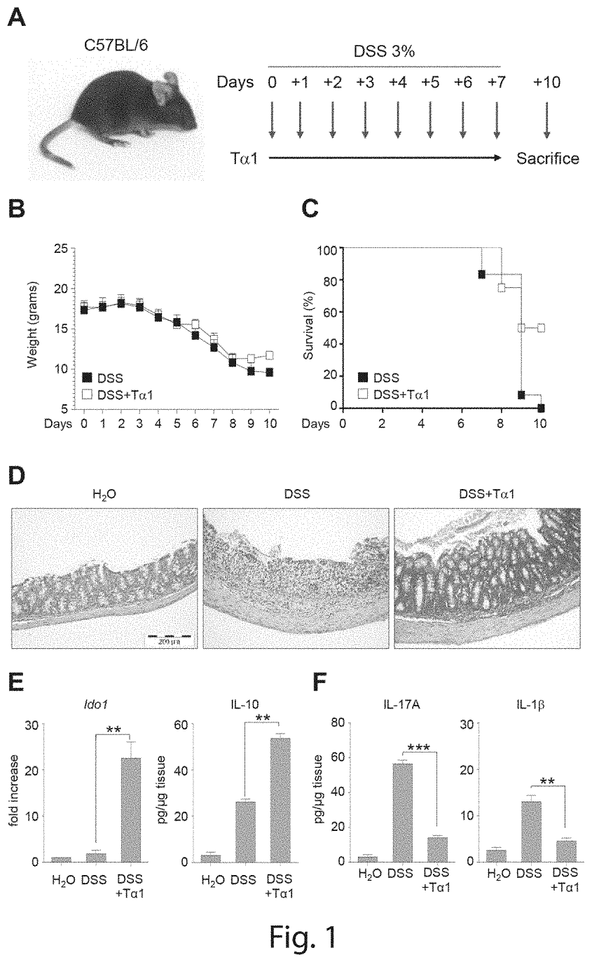

DSS is a water soluble, negatively charged sulfated polysaccharide with a highly variable molecular weight ranging from 5 to 1400 kDa. Murine colitis results from administration of 40-50 kDa DSS added to drinking water. In the DSS model, the sulfated polysaccharide does not directly induce intestinal inflammation, but rather acts as a direct chemical toxin to colonic epithelium resulting in epithelial cell injury. We have added 40-50 kDa DSS to sterilized drinking water at 3% for a period of 6 days to induce acute colitis. Concomitantly, Tα1 at 200 μg/kg was intraperitoneally injected daily, as illustrated in FIG. 1A. Control mice receive vehicle alone. Surviving mice were sacrificed at 10 days after the initiation of colitis.

CIC Model.

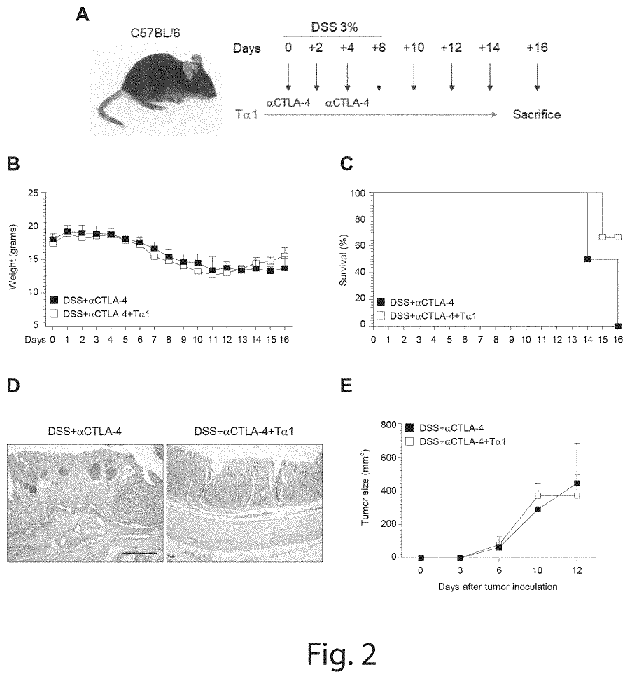

The mice received 3% DSS in their drinking water for 8 days and 100 μg of anti-CTLA-4 mAb (BioXcell, USA) or isotype control antibody intraperitoneally at the beginning of the experiment (day 0) and 4 days after (day +4). Surviving mice were sacrificed at 16 days. Tα1 at 200 μg/kg was intraperitoneally injected every other day, as illustrated in FIG. 2A.

In both models, animals were monitored daily for appearance of diarrhea, fecal blood, loss of body weight and survival. At the end of the experiment, surviving mice were sacrificed, the colon was excised, and evaluated for macroscopic damage and local immune parameters

Tumor Challenge.

B16-F0 (ATCC® CRL-6322™ were cultured in RPMI Medium1640 (Gibco, Life Technologies, USA) containing 10% FBS (Gibco, USA), 100 U/mL penicillin, and 100 μg/mL streptomycin, at 37° C. in a humidified atmosphere of 5% CO2. 2×10⁵ B16 tumor cells were subcutaneously injected into the right flanks of the mice. The mice were injected intraperitoneally with 100 μg of anti-CTLA-4 mAb, at 0, 6, and 10 days post-tumor implantation and concomitantly with Tα1 at 200 μg/kg intraperitoneally. Tumor size was measured with a caliper and calculated as described (Wang et al., 2019).

Colitis Scores and Histologic Analysis.

Freshly isolated colons were fixed in formalin and embedded in paraffin. H&E staining was performed using a standard protocol. For the quantitative histological analysis, five criteria were used to grade each section of the intestine: (i) severity of inflammation, (ii) percent of area affected by inflammation, (iii) degree of hyperplasia, (iv) depth of the lesion, and (v) ulceration.

Immune Assays.

The expression of the IDO1 gene (Ido1) in the colon was assessed by RT-PCR using specific primers (Zelante et al., 2013). The levels of cytokines in the colon homogenates were determined by specific ELISA (R&D Systems).

Statistical Analysis.

Student's t-test, one- or two-way ANOVA with Bonferroni post-hoc test were used to determine the statistical significance. Significance was defined as p<0.05. Data are pooled results (mean±SEM) or representative images from three experiments. GraphPad Prism software 6.01 (GraphPad Software) was used for analysis.

Results

FIG. 1 shows the anti-inflammatory effects of Tal in the most widely used experimental model of colitis that mimics IBD (Eichele and Kharbanda, 2017), namely the dextran sodium sulfate (DSS)-induced colitis (FIG. 1A).

The DSS colitis model in IBD research has advantages over other various chemically induced experimental models due to its rapidity, simplicity, reproducibility and controllability. It has been found that treatment with Tα1 prevented the loss of body weight (FIG. 1B), increased survival of mice (FIG. 1C), ameliorated colon histopathology (FIG. 1D), induced the expression of IDO1 and the production of IL-10 in the colon (FIG. 1E) and decreased the production of pro-inflammatory cytokines, such as IL-1S and IL-17A (FIG. 1F). Considering the positive association between baseline IL-17 levels and development of IBD (Moschen et al., 2019), including severe diarrhea/colitis after ipilimumab treatment (Tarhini et al., 2015), these results indicate that Tα1 can have a curative effect in IBD.

FIG. 2 shows that the curative effects of Tα1 can extend to murine model of CIC.

Although the loss of body weight was not significantly prevented by Tα1 (FIG. 2B), the survival of mice was significantly increased by Tal, being the majority of mice surviving at 16 days post initiation of colitis, at the time at which untreated mice have all died (FIG. 2C). Surviving mice showed recovery of the normal architecture structure of the colon as compared to untreated animals (FIG. 2D). To rule out the possibility that the amelioration of the immunopathology by Tα1 occurs at the cost of antitumoral efficacy, the growth kinetics of established B16 melanoma in these mice were measured. Treatment with Tα1 did not affect the growth kinetics of the tumors afforded by treatment with anti-CTLA-4 Mab (FIG. 2E).

### REFERENCES

The present disclosure includes the following references, which are incorporated herein in their entirety by this reference thereto:
Camerini, R., and Garaci, E. (2015). Historical review of thymosin alpha 1 in infectious diseases. Expert opinion on biological therapy 15 Suppl 1, S117-127.
Darvin, P., Toor, S. M., Sasidharan Nair, V., and Elkord, E. (2018). Immune checkpoint inhibitors: recent progress and potential biomarkers. Experimental & Molecular Medicine 50, 165.
Eichele, D. D., and Kharbanda, K. K. (2017). Dextran sodium sulfate colitis murine model: An indispensable tool for advancing our understanding of inflammatory bowel diseases pathogenesis. World J Gastroenterol 23, 6016-6029.
Fritz, J. M., and Lenardo, M. J. (2019). Development of immune checkpoint therapy for cancer. The Journal of Experimental Medicine 216, 1244-1254.
Garaci, E., Pica, F., Matteucci, C., Gaziano, R., D'Agostini, C., Miele, M. T., Camerini, R., Palamara, A. T., Favalli, C., Mastino, A., et al. (2015). Historical review on thymosin alpha1 in oncology: preclinical and clinical experiences. Expert Opinion on Biological Therapy 15 Suppl 1, S31-39.
Goldstein, A. L., and Goldstein, A. L. (2009). From lab to bedside: emerging clinical applications of thymosin alpha 1. Expert Opinion on Biological Therapy 9, 593-608.
Goldstein, A. L., Guha, A., Zatz, M. M., Hardy, M. A., and White, A. (1972). Purification and biological activity of thymosin, a hormone of the thymus gland. Proceedings of the National Academy of Sciences of the United States of America 69, 1800-1803.
Haanen, J., Carbonnel, F., Robert, C., Kerr, K. M., Peters, S., Larkin, J., Jordan, K., and Committee, E. G. (2018). Management of toxicities from immunotherapy: ESMO Clinical Practice Guidelines for diagnosis, treatment and follow-up. Ann Oncol 29, iv264-iv266.
Karamchandani, D. M., and Chetty, R. (2018). Immune checkpoint inhibitor-induced gastrointestinal and hepatic injury: pathologists' perspective. J Clin Pathol 71, 665-671.
Ladak, K., and Bass, A. R. (2018). Checkpoint inhibitor-associated autoimmunity. Best Pract Res Clin Rheumatol 32, 781-802.
Li, C., Bo, L., Liu, Q., and Jin, F. (2015). Thymosin alpha1 based immunomodulatory therapy for sepsis: a systematic review and meta-analysis. International journal of infectious diseases: IJID: official publication of the International Society for Infectious Diseases 33, 90-96.
Marin-Acevedo, J. A., Chirila, R. M., and Dronca, R. S. (2019). Immune Checkpoint Inhibitor Toxicities. Mayo Clin Proc 94, 1321-1329.
Marin-Acevedo, J. A., Harris, D. M., and Burton, M. C. (2018). Immunotherapy-Induced Colitis: An Emerging Problem for the Hospitalist. J Hosp Med 13, 413-418. Moschen, A. R., Tilg, H., and Raine, T. (2019). IL-12, IL-23 and IL-17 in IBD: immunobiology and therapeutic targeting. Nat Rev Gastroenterology & Hepatology 16, 185-196.
Pardoll, D. M. (2012). The blockade of immune checkpoints in cancer immunotherapy. Nat Rev Cancer 12, 252-264.
Rocha, M., Correia de Sousa, J., Salgado, M., Araujo, A., and Pedroto, I. (2019). Management of Gastrointestinal Toxicity from Immune Checkpoint Inhibitor. GE Port J Gastroenterol 26, 268-274.
Romani, L., Bistoni, F., Perruccio, K., Montagnoli, C., Gaziano, R., Bozza, S., Bonifazi, P., Bistoni, G., Rasi, G., Velardi, A., et al. (2006). Thymosin alpha1 activates dendritic cell tryptophan catabolism and establishes a regulatory environment for balance of inflammation and tolerance. Blood 108, 2265-2274.
Romani, L., Oikonomou, V., Moretti, S., Iannitti, R. G., D'Adamo, M. C., Villella, V. R., Pariano, M., Sforna, L., Borghi, M., Bellet, M. M., et al. (2017). Thymosin alpha1 represents a potential potent single-molecule-based therapy for cystic fibrosis. Nat Med 23, 590-600.
Samaan, M. A., Pavlidis, P., Papa, S., Powell, N., and Irving, P. M. (2018). Gastrointestinal toxicity of immune checkpoint inhibitors: from mechanisms to management. Nature reviews Gastroenterology & Hepatology 15, 222-234.
Tarhini, A. A., Zahoor, H., Lin, Y., Malhotra, U., Sander, C., Butterfield, L. H., and Kirkwood, J. M. (2015). Baseline circulating IL-17 predicts toxicity while TGF-beta1 and IL-10 are prognostic of relapse in ipilimumab neoadjuvant therapy of melanoma. J Immunother Cancer 3, 39.
Wang, T., Zheng, N., Luo, Q., Jiang, L., He, B., Yuan, X., and Shen, L. (2019). Probiotics Lactobacillus reuteri Abrogates Immune Checkpoint Blockade-Associated Colitis by Inhibiting Group 3 Innate Lymphoid Cells.

Frontiers in Immunology 10, 1235.
Wilky, B. A. (2019). Immune checkpoint inhibitors: The linchpins of modern immunotherapy. Immunol Rev 290, 6-23.
Zelante, T., Iannitti, R. G., Cunha, C., De Luca, A., Giovannini, G., Pieraccini, G., Zecchi, R., D'Angelo, C., Massi-Benedetti, C., Fallarino, F., et al. (2013). Tryptophan catabolites from microbiota engage aryl hydrocarbon receptor and balance mucosal reactivity via interleukin-22. Immunity 39, 372-385.

What is claimed is:

1. A method for treatment and/or reduction of occurrence of immune checkpoint inhibitor-related immune adverse effects in a subject in need thereof, comprising administering thymosin alpha 1 to the subject.

2. The method according to claim 1, wherein the immune checkpoint inhibitor-related immune adverse effects are selected from the group consisting of checkpoint inhibitor colitis, diarrhea, rash, elevated alanine amino transferase (ALT), hypothyroidism, and hypophysitis.

3. The method according to claim 1, wherein the thymosin alpha 1 is administered in a pharmaceutical composition that comprises the thymosin alpha 1 as active principle, together with one or more excipients and/or adjuvants.

4. The method according to claim 3, wherein the immune checkpoint inhibitor-related immune adverse effects are selected from the group consisting of checkpoint inhibitor colitis, diarrhea, rash, elevated alanine amino transferase (ALT), hypothyroidism, and hypophysitis.

5. The method according to claim 1, wherein the thymosin alpha 1 is administered in combination with one or more immune checkpoint inhibitors, wherein the thymosin alpha 1 and the one or more immune checkpoint inhibitors are administered simultaneously, separately or sequentially.

6. The method according to claim 5, wherein the immune checkpoint inhibitor-related immune adverse effects are selected from the group consisting of checkpoint inhibitor colitis, diarrhea, rash, elevated alanine amino transferase (ALT), hypothyroidism, and hypophysitis.

7. The method according to claim 5, wherein said one or more immune checkpoint inhibitors are selected from the group consisting of anti CTLA-4, anti PD-1, anti PD-L1, and combinations thereof.
